(19) United States
(12) Patent Application Publication
(10) Pub. No.: US 2019/0218181 A1
(43) Pub. Date: Jul. 18, 2019

(54) PROCESS FOR THE MANUFACTURE OF HYDROXY-SUBSTITUTED AROMATIC COMPOUNDS

(71) Applicant: BASF SE, Ludwigshafen am Rhein (DE)

(72) Inventors: Thomas Markert, Düsseldorf (DE); Ulrich Issberner, Monheim Am Rhein (DE); Markus Dierker, Düsseldorf (DE); Dominik Ohlmann, Ludwigshafen (DE)

(21) Appl. No.: 16/099,462

(22) PCT Filed: May 10, 2017
(86) PCT No.: PCT/EP2017/061156

§ 371 Date: Nov. 7, 2018

(30) Foreign Application Priority Data
May 10, 2016 (EP) 16169014.4

(51) Int. Cl. C07D 213/16; C07C 43/23; C07C 39/21; C07C 215/80; C07C 49/248

(52) U.S. Cl. CPC C07D 213/16; C07C 43/23; C07C 49/248; C07C 215/80; C07C 39/21

(57) ABSTRACT

The present invention relates to a process for the manufacture of hydroxy-substituted aromatic styryl or stilbene compounds.

The present invention relates to a process for the manufacture of hydroxy-substituted aromatic compounds, in particular, aromatic styryl compounds (sometimes also referred to as stilbene or stilbenoid compounds). Hydroxy-substituted aromatic styryl or stilbenoid compounds are known and have been recently attracted attention in the pharmaceutical area. For example, resveratrol

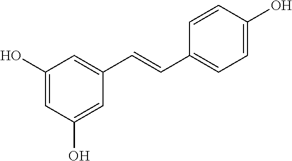 [SMILES: Oc1ccc(/C=C/c2cc(O)cc(O)c2)cc1]

exhibits interesting antioxidant properties (e.g. WO 01/30336). WO 2008/131059 relates to a process of intranasally administering prodrugs of curcumin:

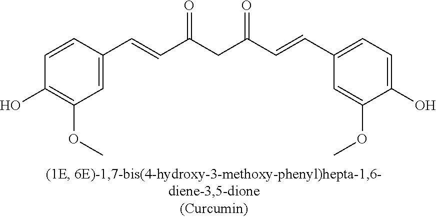 [SMILES: COc1cc(/C=C/C(=O)CC(=O)/C=C/c2ccc(O)c(OC)c2)ccc1O]

curcumin analogs, hybrids of curcumin and various other natural polyphenols, in a bolus of helium gas to treat Alzheimer's disease. While details of the manufacturing methods are not given, in the figures of WO 2008/131059 exemplified methods are shown, which require e.g. the reaction of the corresponding phenols with corresponding aldehydes (e.g. FIG. 3):

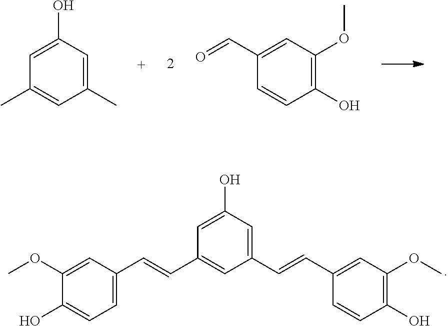

WO 2010/074971 A1 mentions the same methods. Also U.S. Pat. No. 8,758,731 refers to U.S. Pat. No. 7,745,670 (corresponding to WO2008131059A2) with respect to the manufacture of 1-hydroxyl 3,5-bis(4′hydroxyl styryl)benzene. US 2010/0190803 A1 relates to similar compounds of the formula:

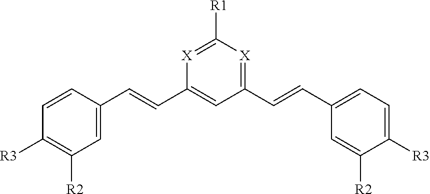

wherein R1, R2 and R3 include inter alia hydroxyl, useful in the treatment of diseases featuring amyloids, such as Alzheimer's disease. The processes of manufacturing these compounds include for the bis(styryl)pyrimidine compounds (X═N) the condensation of the corresponding dimethyl compounds

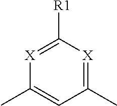

with aldehydes of the formula:

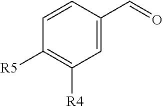

to obtain a compound of the formula:

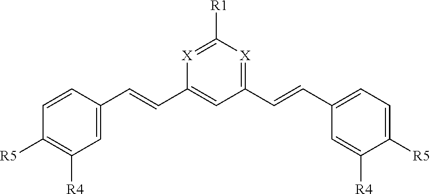

and deprotecting the compound to obtain the target molecule. For the bis(styryl)benzene compounds (X═CH) a benzene compound of formula:

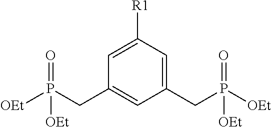

is reacted with a benzaldehyde compound of formula

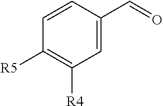

to obtain a compound of the formula:

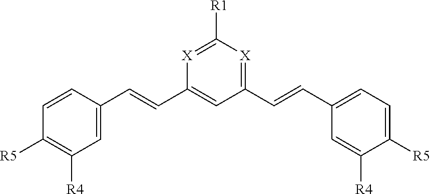

and deprotecting the compound to obtain the target molecule. The process is also described in Bioorganic & Medicinal Chemistry 20 (2012) 4921-4935. This multistage process including the preparation of the starting materials is very costly, so that the process is not suitable for larger scale manufacture. A similar approach is disclosed in NAAMA KARTON-LIFSHIN ET AL, JOURNAL OF THE AMERICAN CHEMICAL SOCIETY, vol. 134, no. 50, 19 Dec. 2012 (2012-12-19), pages 20412-20420.

While palladium-catalyzed Heck coupling between an olefin and aryl halide was also used in the manufacture of biologically active stilbenoids like resveratrol, it was found that the synthetic efficiency for hydroxyl-functionalized stilbenoids was hampered by the involvement of additional protection/ deprotection strategies (see e.g. Angew. Chem. 2012, 124, 12416-12419, which document describes the coupling of 4-iodophenol and acrylic acid in the presence of a palladium catalyst to obtain hydroxylated stilbenoids). Similar, ANGEWANDTE CHEMIE INTERNATIONAL EDITION, vol. 51, no. 49, 3 Dec. 2012 (2012-12-03), pages 12250-12253) does not describe the reaction of p-coumaric acid with a hydroxyl-substituted aryl halid. This applies also for HU KANG ET AL, JOURNAL OF THE AMERICAN CHEMICAL SOCIETY, vol. 129, no. 11, 1 Mar. 2007 (2007-03-01), pages 3267-3286 and ROSA MARTI-CENTELLES ET AL, BIOORGANIC & MEDICINAL CHEMISTRY, vol. 21, no. 11, 1 Jun. 2013 (2013-06-01), pages 3010-3015.

RENE CSUK ET AL, ARCHIV DER PHARMAZIE, vol. 346, no. 7, 30 Jul. 2013 (2013-07-30), pages 499-503, describes the manufacture of resveratrol derivatives.

SHANE SELLARAJAH ET AL, JOURNAL OF MEDICINAL CHEMISTRY, AMERICAN CHEMICAL SOCIETY, US, vol. 47, no. 22, 1 Jan. 2004 (2004-01-01), pages 5515-5534, does not disclose the manufacture of so to say all hydroxyl-substituted compounds. For example, also Chem. Eur. J. 2013, 19, 17980-17988, describes the manufacture of

4,4′,4″-[(1E,1′E,1″E)-Benzene-1,3,5-triyltris(ethene-2,1-diyl)]triphenol (indicated as compound 19) from the corresponding triacetate (14) and not by subjecting the starting 1,3,5-tribromobenzene directly to the reacting with the corresponding hydroxy styrenes. It is therefore not surprising that also e.g. WO 2006/136135, relating to a method for the decarboxylating C═C bond formation by reacting carboxylic salts with carbon electrophiles in the presence of transition metal compounds as catalysts, does not disclose the reaction of any hydroxy-functional compounds. Moreover WO 2006/136135 also does not disclose the reaction of polyfunctional carbon electrophiles which react with more than one mol of the carboxylic acid.

Accordingly, the present inventors searched for a possibility to provide a simple and inexpensive access to hydroxy-substituted aromatic styryl or stilbene compounds.

Surprisingly they found out that hydroxy-substituted aromatic compounds of the formula (I):

Z—(CH═CH—Ar)ₐ (I)

wherein

Z is selected from a diivalent substituted aromatic group, or a divalent group of the formula:

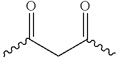

(wherein [symbol] denotes a single bond),

Ar independently is selected from substituted aromatic groups, and

a is 2,

or a salt thereof,

wherein both of the groups Z and Ar are substituted with at least one hydroxy group, can be obtained in high yields with a much shorter synthetic route than described for example in US 2010/0190803 A1, and which synthetic route also does not require the costly introduction of any hydroxyl protective groups. Therefore, the process according to the present invention is also suitable for the production of these compounds on an industrial scale.

Accordingly, the present invention provides a process for the manufacture of hydroxy-substituted aromatic compounds of the formula (I):

Z—(CH═CH—Ar)ₐ (I)

wherein

Z is selected from a divalent substituted aromatic group, or a divalent group of the formula:

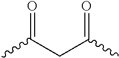

(wherein [symbol] enotes a single bond),

Ar independently is selected from substituted aromatic groups, and

a is 2,

or a salt thereof,

which comprises reacting a compound of the formula (II):

Z—(X)ₐ (II)

wherein

X is a leaving group, preferably a halogenide group, and

Z and a are as defined above,

with a compound of the formula (III):

CH₂═CH—Ar (III)

wherein Ar is as defined above,

in the presence of a transition metal catalyst, with the proviso that the groups Z and Ar are each substituted with at least one hydroxy group. In accordance with the present invention the term “substituted with at least one hydroxy group” is intended to mean that the hydroxyl group is directly attached to the aromatic groups of Z or Ar via its oxygen atom.

Z can only carry a hydroxyl substituent group in case it is a divalent substituted aromatic group, i.e. the residue Z being a divalent group of the formula:

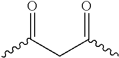

does not carry a hydroxyl substituent.

In the present invention the group Z is a divalent substituted aromatic group (a being 2). Throughout the invention, the term “optionally substituted mono-, di or trivalent aromatic group” shall include carbocyclic aromatic groups (wherein the aromatic ring system is formed of carbon atoms) and heteroaromatic groups (wherein the aromatic ring system is formed of carbon atoms and at least one heteroatom. As explained before, there is at least one hydroxy group as substituent on Z and Ar.

Mono-, di or trivalent carbocyclic aromatic groups (sometimes referred to as aryl groups) may be formally derived from the corresponding aromatic hydrocarbon compounds containing preferably 6 to 14 carbon atoms (excluding the carbon atoms of the possible substituents), which may be monocyclic or bicyclic, preferably monocyclic. Such compounds from which the corresponding monovalent, divalent or trivalent groups are formally derived from, include for example benzene (i.e. phenyl or phenylene or benzene-tri-yl), naphthalene, anthracene and phenanthrene.

The aforementioned aryl groups may have one or more, preferably 1 to 3, more preferably 1 or 2 of the same or different substituents, even more preferred 1 substituent, which optionally may have up to 10 carbon atoms, and which is in particular selected from halogen, such as preferably F and Cl, cyano, optionally substituted alkyl, such as preferably methyl, ethyl, n-propyl, i-propyl, halogen-substituted alkyl such as trifluoromethyl, hydroxy-substituted alkyl such as hydroxymethyl, aminocarbonyl-substituted alkyl such as aminocarbonylmethyl, carboxyl-substituted alkyl such as carboxymethyl, an alkenyl group such as propenyl, optionally substituted alkoxy, such as preferably methoxy and ethoxy, a hydroxyl group (—OH), a carboxyl group [—(C═O)—OH], a heterocyclyl group, such as a N-morpholinyl group, an aminocarbonyl group, an optionally substituted amino group, such as preferably amino (NH₂—) or mono- or di-alkylamino such as preferably dimethylamino, an optionally substituted acyl group such as formyl or acetyl. The most preferred substituent group is hydroxyl, even more one (1) hydroxyl group. Optionally substituted phenyl (a=1) or phenylene (a=2) or benzene-tri-yl (a=3) is preferred as Z. More preferred aryl groups for Z are phenyl or phenylene each having at least one hydroxyl substituent group. More preferred Z is a phenylene group having one (1) hydroxyl substituent, e.g.:

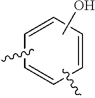

wherein each [symbol] denotes a single bond. Most preferred Z is:

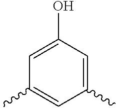

wherein each [symbol] denotes a single bond.

Divalent optionally substituted heteroaromatic groups (sometimes referred to as heteroaryl groups) as groups Z may be formally derived from the corresponding heteroaromatic hydrocarbon compounds containing preferably 4 to 9 ring carbon atoms, which additionally preferably contain 1 to 3 of the same or different heteroatoms from the series S, O, N, preferably N, in the ring and therefore preferably form 5- to 12-membered heteroaromatic residues which may preferably be monocyclic but also bicyclic. Preferred aromatic heterocyclic residues (that may be also di- or trivalent by formally removing one or two further hydrogen atom) include: pyridyl (pyridinyl), pyridyl-N-oxide, pyridazinyl, pyrimidyl, pyrazinyl, thienyl (thiophenyl), furyl, pyrrolyl, pyrazolyl, imidazolyl, triazolyl, thiazolyl, oxazolyl or isoxazolyl, indolizinyl, indolyl, benzo[b]thienyl, benzo[b]furyl, indolyl, quinolyl, isoquinolyl, naphthyridinyl, quinazolinyl, quinoxalinyl etc.

The aforementioned heteroaryl-groups may have one or more, preferably 1 to 3, more preferably 1 or 2 same or different substituents, even more preferred 1 substituent, which are in particular selected from halogen, such as preferably F and Cl, cyano, optionally substituted alkyl as defined below, such as preferably methyl, ethyl, n-propyl, i-propyl, halogen-substituted alkyl such as trifluoromethyl, hydroxy-substituted alkyl such as hydroxymethyl, aminocarbonyl-substituted alkyl such as aminocarbonylmethyl, carboxyl-substituted alkyl such as carboxymethyl, an alkenyl group such as propenyl, optionally substituted alkoxy, such as preferably methoxy and ethoxy, optionally substituted alkylthio, such as methylthio, a hydroxyl group (—OH), an oxo-group (═O), a carboxyl group [—(C═O)—OH], a heterocyclyl group as defined above, such as a N-morpholinyl group, an aminocarbonyl group, an optionally substituted amino group, such as preferably amino (NH₂—) or mono- or di-alkylamino such as preferably dimethylamino, with the proviso that there is at least one hydroxyl group, even more preferred one (1) hydroxyl group.

Preferred groups Z are in particular divalent pyridyl or pyrimidinyl groups, which preferably have at least one, preferably one (1) hydroxyl group of the formula:

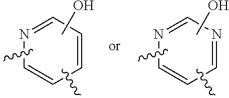

wherein each [symbol] denotes a single bond. More preferred are pyrimidinyl groups of the formula:

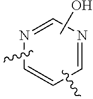

and even more of the formula:

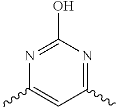

wherein each [symbol] denotes a single bond.

In a further preferred embodiment of the invention the group Z may be also a group of the formula:

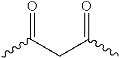

wherein each [symbol] denotes a single bond.

It goes without saying that such group does not carry a hydroxyl substituent.

In the present invention the groups Ar (since a=2, there are two Ar groups) can be the same or different and are independently selected from optionally substituted aromatic groups, which can be selected from the same groups as mentioned for the monovalent groups above. Among them the most preferred group Ar is an optionally substituted phenyl group that may have 1 to 3, preferably 1 to 2, even more preferred one (1) substituent groups, which optionally may have up to 6 carbon atoms and are preferably selected from hydroxyl, alkoxy, such as methoxy, or ethoxy, optionally substituted alkylthio, such as methylthio, amino (—NH₂), mono or di(alkyl or aryl) amino, such as dimethylamino, with the proviso that each Ar carries at least one hydroxyl group. There are two Ar groups (a=2) and preferably these Ar groups are identical. More preferred Ar is a phenyl group that carries at least one, more preferred one (1) hydroxyl group, and optionally one (1) further substituent group, like C1-C6 alkoxy or di(C1-C6)alkylamino. Most preferred Ar is phenyl group having one (1) hydroxyl group.

Depending on the substituent groups of the groups Z and Ar, the compounds of formula (I) of the present invention may be easily transformed into their corresponding salts with acids to form, for example, salts with corresponding anions, such as carboxylates, sulfonates, sulfates, chloride, bromide, iodide, phosphate, tartrates, methanesulfonate, hydroxyethanesulfonate, glycinate, maleate, propionate, fumarate, tulouenesulfonate, benzene sulfonate, trifluoroacetate, naphthalenedisulfonate-1,5, salicylate, benzoate, lactate, salts of malic acid, salts of 3-hydroxy-2-naphthoic acid-2, citrate and acetate, or with bases, to form, for example, salts with alkaline or alkaline-earth hydroxides, such as NaOH, KOH, Ca(OH)₂, Mg(OH)₂ etc., amine compounds such as ethylamine, diethylamine, triethylamine, ethyldiisopropylamine, ethanolamine, diethanolamine, triethanolamine, methylglucamine, dicyclohexylamine, dimethylaminoethanol, procaine, dibenzylamine, N-methylmorpholine, arginine, lysine, ethylenediamine, N-methylpiperidin, 2-amino-2-methyl-propanol-(1), 2-amino-2-methyl-propandiol-(1,3), 2-amino-2-hydroxyl-methyl-propandiol-(1,3) (TRIS) etc. Such salt formation includes also salts of bases with the acidic phenolic hydroxyl groups. Amino groups as substituent groups of Z and Ar allow in particular for salt formation with acids.

Depending on their structure, the compounds prepared according to the process of the invention may exist in stereoisomeric forms (enantiomers, diastereomers) in the presence of asymmetric carbon atoms. The invention therefore includes the use of the enantiomers or diastereomers and the respective mixtures thereof. The pure enantiomer forms may optionally be obtained by conventional processes of optical resolution, such as by fractional crstallisation of diastereomers thereof by reaction with optically active compounds. Since the compounds according to the invention may occur in tautomeric forms, the present invention covers the use of all tautomeric forms.

The compounds provided according to the process of the invention may be present as mixtures of various possible isomeric forms, in particular of stereoisomers such as, for example, E- and Z-, syn and anti, as well as optical isomers. All isomeric forms, including the E-isomers and also the Z-isomers as well as the optical isomers and any mixtures of these isomers are claimed herewith.

The leaving group X used in accordance with the present invention is selected preferably from conventional leaving groups such as —OSO₂R^{F} (perfluoroalkylsulfonates (e.g. triflate)), R-OTs, R-OMs, etc. (tosylates, mesylates), halogenides such as I (iodide), Br (bromide), Cl (chloride), and F (fluoride) etc. More preferred are halogenides, most preferred is bromide, that is compounds Z—(X)ₐ of formula (II) are preferably dibromo substituted aromatic compounds, having preferably at least one, more preferred one (1) hydroxyl group such as, 2,3-dibromophenol (1,2-dibromo-3-hydroxybenzene), 2,4-dibromophenol (1,3-dibromo-6-hydroxybenzene), 2,5-dibromophenol (1,4-dibromo-2-hydroxybenzene), 2,6-dibromophenol (1,3-dibromo-2-hydroxybenzene), 3,4-dibromophenol (1,2-dibromo-4-hydroxybenzene), 3,5-dibromophenol (1,3-dibromo-5-hydroxybenzene). 3,5-dibromophenol is the most preferred compound of formula (II).

Z is selected from a divalent substituted aromatic groups having at least one hydroxyl group. In a preferred embodiment the compounds of formula (I) are triphenols having one hydroxyl group on Z and one hydroxyl group on each group Ar (i.e. a=2).

As the transition metal catalyst conventional catalysts used for coupling reactions, like the heck-type reaction may be used, The most common coupling catalysts are based on palladium, but other transition metals catalysts such as those based on nickel, copper, platinum, iron, cobalt, rhodium, silver, ruthenium may be used as well. It is in particular preferred to use a mixture of catalysts including at least two, preferably exactly two transition metals. The metal can be used in elemental form, as a complex or as a salt. Frequently the metal is introduced as a salt together with a ligand such as phosphines (IUPAC name: phosphanes), amines, N-heterocyclic carbenes, nitriles, and olefins and the catalytic active species is a complex formed in situ from the salt and the ligand.

Preferred palladium catalysts are Pd⁰-catalysts which are frequently prepared in situ from Pd^{II}-salts like Pd(II)-chloride (PdCl₂), Pd(II)-acetate (Pd(OAc)₂), palladium(II)-acetylacetonate, or from activated palladium such e.g. 5% on charcoal and ligands such as phosphines (IUPAC name: phosphanes), like trialkyl- or triaryl phospines such as triphenyl phosphine, or bidentate phosphines like bis(diphenylphosphino)methane, 1,2-bis(diphenylphosphino)-ethane, 1,3-bis(diphenylphosphino)ethane, 1,1′-bis(diphenylphosphino)ferrocene, P(p-MeOPh)₃, tricyclohexylphosphine, tri(o-tolyl)phosphine, P(i-propyl)Ph₂, amines, like bipyridine, 4,4′-dimethyl-2,2′-dipyridyl, phenanthroline (i.e. 1,10-phenanthroline), N-heterocyclic carbenes, nitriles, and olefins. Also chiral ligands such BINAP, TMBTP, Diop, BITIANP, t-Bu-PHOX ((S)-4-tert-butyl-2-[2-(diphenylphosphino)-phenyl]-2-oxazoline) etc. can be used. Preferred are amine ligands. These ligands can be also used if salts of the other transition metals, like nickel, copper, platinum, iron, cobalt, rhodium, silver, ruthenium are applied. Palladium catalysts are the most preferred coupling catalysts used in accordance with the present invention.

In a preferred embodiment the reaction is carried out in the absence of triphenylphosphine leading to triphenyl phosphine oxide which is difficult to be separated from the product of formula (I), more preferably the reaction is carried out in the absence of any phosphines.

In a preferred embodiment of the process according to the invention the compound of formula (III)

CH₂═CH—Ar (III)

is formed in situ from a compound of formula (IV)

HOOC—CH═CH—Ar (IV)

wherein Ar is as defined above. In principle it is possible to use also salts of the compound of formula (IV), for example with bases, like alkaline or earth alkaline metal oxides, hydroxides, carbonates, bicarbonates, and carboxylates, like in particular acetates. But preferably the carboxylic acids of formula (IV) are added to the reaction mixture as such. In practicing this most preferred embodiment, instead of reacting the in particular hydroxyl substituted styryl derivatives of formula (III), the corresponding in particular hydroxyl-substituted cinnamic acid derivatives of formula (IV) are reacted with the in particular hydroxyl-substituted electrophiles of formula (II). This embodiment turned out to be most preferably, because in particular, the hydroxyl-substituted styryl derivatives of formula (III) turned out to be potential subject to various side reactions, such as polymerization reactions, which diminishes the yield of the coupling reaction. Surprisingly the corresponding, in particular, hydroxyl-substituted cinnamic acid derivatives of formula (IV) can be subjected to the decarboxylative cross-coupling reaction with the, in particular, hydroxyl substituted electrophiles of formula (II) with high yields even at large scales. While the decarboxylative cross-coupling reaction in principle was known (see e.g. Wikipedia on keyword “decarboxylative cross-coupling” and references cited therein; Nuria Rodriguez and Lukas J. Goossen, Chem. Soc. Rev., 2011, 40, 5030-5048 Decarboxylative coupling reactions: a modern strategy for C—C-bond formation; WO 2006/136135) it was not known for the reaction of cinnamic acid derivatives of formula (IV) with the electrophiles of formula (II), wherein at least one or both of the compounds of formula (IV) and (II) carry a hydroxyl substituent. In carrying out the decarboxylative cross-coupling reaction in principle known catalyst systems can be used, such as those described in the aforementioned three documents on decarboxylative cross-coupling reactions. For example basically copper monometallic systems (e.g. using Cu(I)-compounds such as Cu(I)-oxide, Cu(I)-halogenides such as iodides or bromides, or using Ag(I)-compounds such as Ag(I)-oxide, Ag(I)-halogenides such as iodides or bromides, Ag₂CO₃), and optionally ligands such as amines like phenanthroline) can be used. Further palladium monometallic systems, using Pd(II)-salts, such as Pd(II)-acetate in the presence of ligands such as phosphines can be used. In a preferred embodiment of this the decarboxylative cross-coupling of compounds of formula

HOOC—CH═CH—Ar (IV)

a catalyst system comprising two transition metals is used. Without being bond to theory, in such catalyst system one transition metal (like for example copper or silver) is involved in the decarboxylation reaction and the other transition metal (like in particular palladium) is involved in the coupling of the resulting decarboxylated compound. The present invention includes both, the initial separate decarboxylation of the compounds of formula (IV), in particular, with a copper-based catalyst preferably in the presence of an amine ligand, such as 1,10-phenanthroline, isolation of the corresponding styryl compounds

H₂C═CH—Ar (IV′)

and subsequently the coupling with a compound of formula (II) in the presence of a palladium catalyst, and the simultaneous decarboxylative coupling of the compound of formula (IV) with a compound of formula (II), in particular, in the presence of a bimetallic catalyst. Such bimetallic catalyst systems comprising two transition metals include for example palladium- copper or palladium-silver bimetallic systems.

In a most preferred embodiment of the invention, a palladium-copper catalyst system is used.

In this embodiment preferably a Pd(II)-salt such as Pd(II)-acetate (Pd(acetate)₂), Pd(II)-chloride (PdCl₂), Pd(II)-acetylacetonate (Pd(acac)₂) and a Cu(II)-salt or a Cu(I)-salt, such as Cu(OH)₂, CuCO₃, CuI, CuBr, CuCl are reacted in the presence of at least one ligand as the above mentioned ligands, such as phosphines and/or amines, preferably in the presence of both, at least one amine and at least one phosphine (phosphane), preferably an aromatic amine and a tris(aryl)phosphine. In the most preferred embodiment the catalyst system used in particular in the decarboxylative cross-coupling reaction of the in particular hydroxyl-substituted cinnamic acid derivatives of formula (IV) and the in particular hydroxyl-substituted electrophiles of formula (II) comprises a Pd(II)-salt, a Cu(II)-salt and at least one ligand selected from amines and phospanes, which are most preferably phenanthroline (i.e. 1,10-phenanthroline) and a triarylphosphine, in particular, triphenyl phosphine.

As the transition metal catalyst the palladium(0)-compounds can be also directly used (i.e. without their in situ formation), preferred are palladium(0)-bis(phosphines), in particular palladium(0)-bis(triphenylphosphine).

In a preferred embodiment of the invention the transition metal catalyst i.e. the transition metal catalyst system is used in concentrations related to the total amount of the metal(s) contained in such transition metal catalyst system for example in the range between 0 and 15 mol %, preferably 2 to 12 mol % based in particular on the molar amount of the compound of formula (II). In the case of the preferred catalyst systems comprising two transition metals the amount of the metal supposed to be involved in decarboxylation reaction (like for example copper or silver, preferably copper) is for example in the range of between 0 and 15 mol %, preferably 2 to 12 mol % based in particular on the molar amount of the compound of formula (II), and the amount of the metal supposed to be involved in the coupling reaction, like in particular palladium, is between 0 and 1 mol % preferably between 0.01 to 0.5 mol % (mol % shall relate here to the amount of metal, i.e. one mol of copper relates to 63.546 g, and one mol of palladium relates to 106.42 g).

In a most preferred embodiment at least one copper(II)salt and 1,10-phenanthroline is used, more preferred in combination with at least one palladium compound, preferably a palladium(II)-salt but no phosphine ligand.

In a preferred embodiment of the process according to the invention the substituent groups Z and Ar at the CH═CH-group of formula (I) take the trans-positions, i.e.:

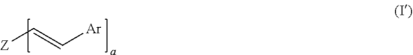

In a preferred embodiment of the process according to the invention the leaving group X is selected from halogenides, preferably chlorine and bromine, more preferably bromine.

In a further preferred embodiment of the process according to the invention Z is a substituted divalent six-membered aromatic group, preferably selected from preferably divalent residues derived from benzene, pyridine, and pyrimidine. With respect to the substituent groups on the six-membered aromatic group it can be referred to the above or the below explanations. A mandatory substituent group is a hydroxyl group. In a still more preferred embodiment of the invention Z is derived from a substituted divalent benzene group. Z is derived from a hydroxyl-substituted divalent preferably divalent benzene group, carrying at least one hydroxyl group preferably exactly one hydroxyl group directly bond to the benzene moiety via the oxygen atom of the hydroxyl group (—OH).

In the preferred embodiment of the invention each group Ar in the general formula (I) or (I′) is derived from a hydroxyl-substituted benzene group, carrying at least one hydroxyl group, preferably exactly one hydroxyl group directly bond to the benzene moiety via the oxygen atom of the hydroxyl group (—OH).

Preferably the present invention the optionally substituent groups of the groups Z and Ar are independently selected from 1 to 3 substituents selected from the group consisting of optionally protected hydroxy, optionally substituted alkyl, optionally substituted alkoxy, optionally substituted alkylthio, optionally substituted acyloxy, optionally substituted amino, like mono- or dialkylamino, again with the proviso that each of the groups that Z and Ar have at least one hydroxyl, preferably exactly one hydroxyl group.

Preferred compounds of formula (I) that can be obtained according to the process of the present invention are as follows:

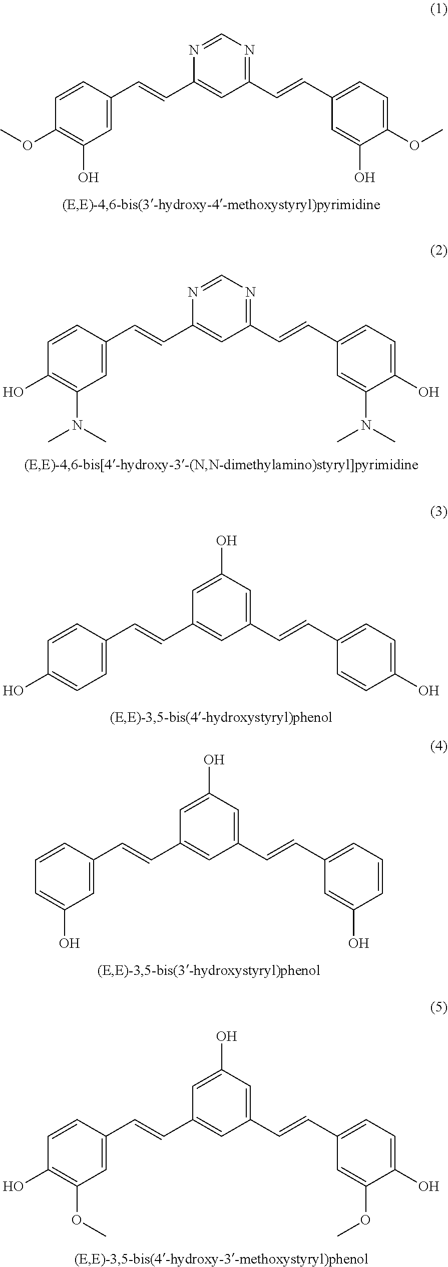

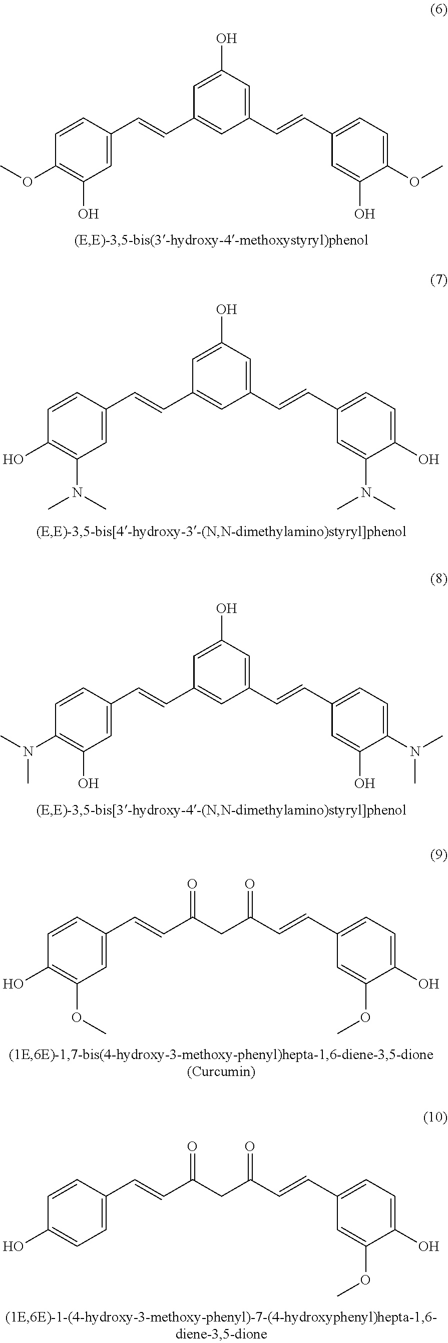

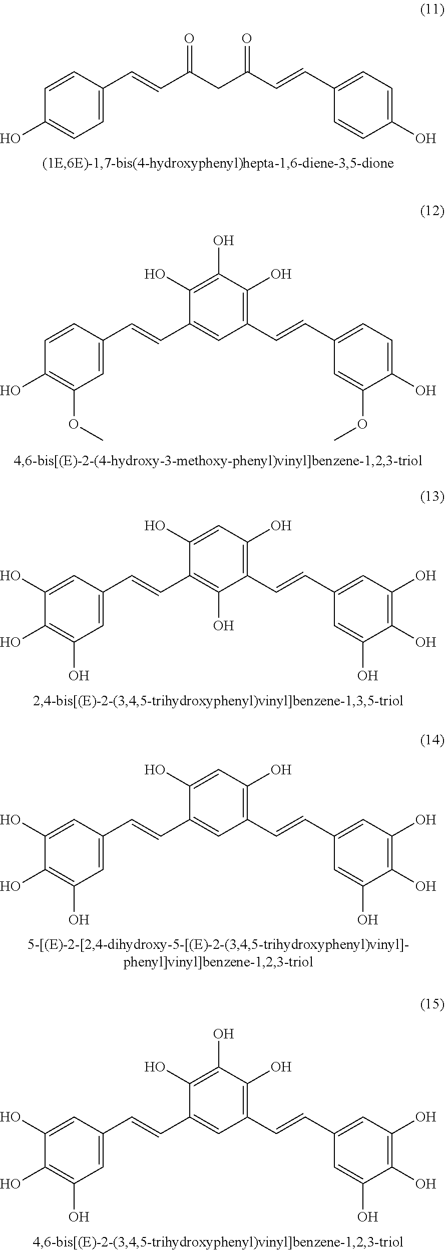 [SMILES: O=C(/C=C/c1ccc(O)cc1)CC(=O)/C=C/c1ccc(O)cc1 ; COc1cc(/C=C/c2cc(/C=C/c3ccc(O)c(OC)c3)c(O)c(O)c2O)ccc1O ; Oc1cc(/C=C/c2c(O)cc(O)c(/C=C/c3cc(O)c(O)c(O)c3)c2O)cc(O)c1O ; Oc1cc(O)c(/C=C/c2cc(O)c(O)c(O)c2)cc1/C=C/c1cc(O)c(O)c(O)c1 ; Oc1cc(/C=C/c2cc(/C=C/c3cc(O)c(O)c(O)c3)c(O)c(O)c2O)cc(O)c1O]

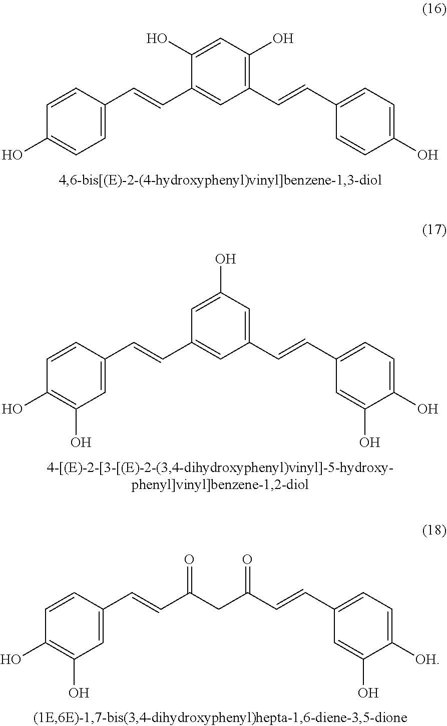 [SMILES: Oc1ccc(/C=C/c2cc(/C=C/c3ccc(O)cc3)c(O)cc2O)cc1 ; Oc1cc(/C=C/c2ccc(O)c(O)c2)cc(/C=C/c2ccc(O)c(O)c2)c1 ; O=C(/C=C/c1ccc(O)c(O)c1)CC(=O)/C=C/c1ccc(O)c(O)c1]

The most preferred compound prepared according to the process of the invention is the compound of the formula:

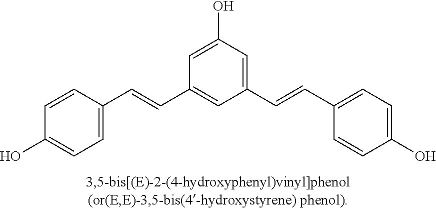 [SMILES: Oc1ccc(/C=C/c2cc(O)cc(/C=C/c3ccc(O)cc3)c2)cc1]

Preferably the process according to the invention is performed in the presence of a solvent, but it may be also carried out without a solvent. Suitable solvents include in particular water, linear, cyclic and branched hydrocarbons (for example hexanes, heptanes and octanes), aromatic hydrocarbons (for example benzene, toluene, xylenes, ethylbenzene, mesitylene), ethers (for example 1,4-dioxane, tetrahydrofuran, methyltetrahydrofuran, dibutyl ether, methyl t-butyl ether, diisopropyl ether, diethylene glycol dimethyl ether, dipropylene glycol), polyethers such as polyalkylene glycols, such as polyethylene glycol (PEG) or polypropylene glycol, esters (for example ethyl acetate, butyl acetate), amides (for example dimethylformamide, diethylformamide, N-methylpyrrolidone, dimethylacetamide), dimethyl sulfoxide, sulfolane, acetonitrile, isobutyronitrile, propionitrile, propylene carbonate and chlorinated aliphatic and aromatic hydrocarbons. It is preferred to use a mixture of water and at least one organic solvent. More preferred is dimethylformamide, diethylformamide, N-methylpyrrolidone, dimethylacetamide, dimethyl sulfoxide, sulfolane, acetonitrile and propylene carbonate. Still more preferred is at least one solvent selected from the group, consisting of N-methyl-2-pyrrolidone (NMP), polyethylene glycol (PEG), acetonitrile, water and dimethylformamide (DMF). Still more preferred is at least one solvent selected from the group consisting of N-methyl-2-pyrrolidone (NMP), dimethylformamide, acetonitrile and water, or mixtures thereof. The most preferred embodiment of the invention a mixture of water, acetonitrile and dimethylformamide is used.

The process according to present invention is preferably carried out in the presence of at least one base, which serves in particular as a scavenger for the leaving group X as mentioned above. Suitable bases include for example inorganic or organic bases, like for example alkaline or earth alkaline oxides, hydroxides, carbonates, bicarbonates, carboxylates, like in particular acetate, and alkoxides, ammonia and organic bases like in particular amines such as mono or dialkylamines, alicyclic or aromatic amines.

The process according to the present invention is preferably carried out at a temperature of at least 80° C., more preferably in a range between 80° C. to 200° C.

In a further embodiment of the present invention the process according to the invention further comprises at least one subsequent derivatization reaction of the compound of formula (I), which is preferably selected from the group consisting of hydrogenation, esterification, etherification, and salt formation, preferably hydrogenation. For example the process according to the invention further comprises at least one hydrogenation reaction to form hydrogenated derivatives of the formula:

Z—(CH₂—CH₂—Ar)ₐ (I″)

wherein Z, Ar and a are as defined above. Such process is carried out in the presence of conventional hydrogenation catalysts such as those based on platinum, palladium, rhodium, ruthenium, and nickel.

In a further preferred embodiment of the invention the process according to invention further comprises the admixture of a compound as obtained in any of these claims with at least one pharmaceutical or cosmetic excipient. Such pharmaceutical or cosmetic excipients include conventional ones, such as saccharose, starch, mannitol, sorbitol, lactose, glucose, cellulose, talcum, calcium phosphate, calcium carbonate; binding agents, such as cellulose, methylcellulose, hydroxypropylcellulose, polypropyl pyrrolidone, gelatine, gum arabic, polyethylene glycol, saccharose, starch; disintegrating agents, such as starch, hydrolyzed starch, carboxymethylcellulose, calcium salt of carboxymethylcellulose, hydroxypropyl starch, sodium glycol starch, sodium bicarbonate, calcium phosphate, calcium citrate; lubricants, such as magnesium stearate, talcum, sodium laurylsulfate; flavorants, such as citric acid, menthol, glycine, orange powder; preserving agents, such as sodium benzoate, sodium bisulfite, paraben (for example methylparaben, ethylparaben, propylparaben, butylparaben); stabilizers, such as citric acid, sodium citrate, acetic acid and multicarboxylic acids from the titriplex series, such as, for example, diethylenetriaminepentaacetic acid (DTPA); suspending agents, such as methycellulose, polyvinyl pyrrolidone, aluminum stearate; dispersing agents; diluting agents, such as water, organic solvents; waxes, fats and oils, such as beeswax, cocoa butter; polyethylene glycol; white petrolatum; etc..

In the following, the preferred embodiments of the invention are summarized:

### Embodiment 1

A process for the manufacture of a hydroxy-substituted aromatic compound of the formula (I):

Z—(CH═CH—Ar)ₐ (I)

wherein

Z is selected from a divalent optionally substituted aromatic group, or a divalent group of the formula:

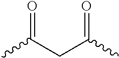

(wherein [symbol] denotes a single bond),

Ar independently is selected from optionally substituted aromatic groups, and

a is 2,

or a salt thereof,

which comprises reacting a compound of the formula (II):

Z—(X)ₐ (II)

wherein

X is a leaving group, preferably a halogenide group, and

Z and a are as defined above,

with a compound of the formula (III):

CH₂═CH—Ar (III)

wherein Ar is as defined above,

in the presence of a transition metal catalyst,

with the proviso that the group Z and the group Ar each are substituted by at least one hydroxy group.

### Embodiment 2

A process according to embodiment 1, wherein Z is selected from a divalent optionally substituted aromatic group.

### Embodiment 3

A process according to any of the previous embodiments, wherein the compound of formula (III) is formed in situ from a compound of formula (IV)

HOOC—CH═CH—Ar (IV)

wherein Ar is as defined above.

### Embodiment 4

A process according to any of the previous embodiments, wherein the transition metal of the transition metal catalyst is selected from the group consisting of palladium nickel, copper, platinum, iron, cobalt, rhodium, silver, ruthenium, and mixtures thereof.

### Embodiment 5

A process according to any of the previous embodiments, preferably according to embodiment 5, wherein the transition metal catalyst is selected from bimetallic catalysts comprising palladium and at least one further transition metal.

### Embodiment 6

A process according to any of the previous embodiments, wherein the transition metal catalyst is selected from transition metal salts, such as halogenides, preferably chlorides, hydroxides, acetates, and trifluoroactetates.

### Embodiment 7

A process according to the previous embodiment 6, wherein at least one ligand for the transition metal, preferably selected from amine ligands and phosphine (or phosphane) ligands, more preferably selected from amine ligands is added.

### Embodiment 8

A process according to any of the previous embodiments, which is carried out in the absence of a phosphine (or phosphane) ligand.

### Embodiment 9

A process according to any of the previous embodiments, wherein at least one amine ligand is added.

### Embodiment 10

A process according to any of the previous embodiments, preferably according to any of the previous embodiments, wherein the transition metal catalyst is selected from bimetallic catalysts comprising palladium and at least one further transition metal selected from copper and silver, preferably copper.

### Embodiment 11

A process according to embodiment 3, wherein the in situ formation of the compound of formula (III) is catalyzed in particular by a copper or silver catalyst, preferably a copper(II)-1,10 phenanthroline catalyst.

### Embodiment 12

A process according to any of the previous embodiments, wherein the transition metal catalyst is selected from palladium(II)-salts, such as palladium(II)-chloride, palladium(II)-acetate, palladium(II)-trifluoroacetate, bis(triphenylphosphine)palladium(II)-chloride, palladium(0)- compounds, preferably palladium(0)-phosphine compounds, such as palladium bis(triphenylphosphine).

### Embodiment 13

A process according to any of the previous embodiments, wherein the transition metal catalyst is used in concentrations related to the amount of the metal between 0 and 15 mol %, preferably 2-12 mol % based on the molar amount of the compound of formula (II).

### Embodiment 14

A process according to any of the previous embodiments, wherein the substituents Z and Ar at the CH═CH-group take the trans-positions.

### Embodiment 15

A process according to any of the previous embodiments, wherein the leaving group X is selected from halogenides, preferably chlorine and bromine, more preferably bromine.

### Embodiment 16

A process according to any of the previous embodiments, wherein Z is an hydroxy-substituted six-membered aromatic group, preferably selected from benzene, pyridine, and pyrimidine.

### Embodiment 17

A process according to any of the previous embodiments, wherein Z is an hydroxyl-substituted benzene group.

### Embodiment 18

A process according to any of the previous embodiments, wherein Z is a hydroxy-substituted benzene group.

### Embodiment 19

A process according to any of the previous embodiments, wherein each group Ar is a hydroxy-substituted benzene group.

### Embodiment 20

A process according to any of the previous embodiments, wherein the groups Z and Ar apart from the hydroxy-group may independently have 1 to 3 substituents selected from the group consisting of protected hydroxy, optionally substituted alkyl, optionally substituted alkoxy, optionally substituted alkylthio, optionally substituted acyloxy, optionally substituted amino, like mono- or dialkylamino.

### Embodiment 21

A process according to any of the previous embodiments, wherein the compound of formula (I) are selected from the group consisting of the compounds

(1) (E,E)-4,6-bis(3′-hydroxy-4′-methoxystyryl)pyrimidine

(2) (E,E)-4,6-bis[4′-hydroxy-3′-(N,N-dimethylamino)styryl]pyrimidine

(3) (E,E)-3,5-bis(4′-hydroxystyryl)phenol

(4) (E,E)-3,5-bis(3′-hydroxystyryl)phenol

(5) (E,E)-3,5-bis(4′-hydroxy-3′-methoxystyryl)phenol

(6) (E,E)-3,5-bis(3′-hydroxy-4′-methoxystyryl)phenol

(7) (E,E)-3,5-bis[4¹-hydroxy-3′-(N,N-dimethylamino)styryl]phenol

(8) (E,E)-3,5-bis[3¹-hydroxy-4′-(N,N-dimethylamino)styryl]phenol

(9) (1E,6E)-1,7-bis(4-hydroxy-3-methoxy-phenyl)hepta-1,6-diene-3,5-dione

(10) (1E,6E)-1-(4-hydroxy-3-methoxy-phenyl)-7-(4-hydroxyphenyl)hepta-1,6-diene-3,5-dione

(11) (1E,6E)-1,7-bis(4-hydroxyphenyl)hepta-1,6-diene-3,5-dione

(12) 4,6-bis[(E)-2-(4-hydroxy-3-methoxy-phenyl)vinyl]benzene-1,2,3-triol

(13) 2,4-bis[(E)-2-(3,4,5-trihydroxyphenyl)vinyl]benzene-1,3,5-triol

(14) 5-[(E)-2-[2,4-dihydroxy-5-[(E)-2-(3,4,5-trihydroxyphenyl)vinyl]-phenyl]vinyl]benzene-1,2,3-triol

(15) 4,6-bis[(E)-2-(3,4,5-trihydroxyphenyl)vinyl]benzene-1,2,3-triol

(16) 4,6-bis[(E)-2-(4-hydroxyphenyl)vinyl]benzene-1,3-diol

(17) 4-[(E)-2-[3-[(E)-2-(3,4-dihydroxyphenyl)vinyl]-5-hydroxy-phenyl]vinyl]benzene-1,2-diol

(18) (1E,6E)-1,7-bis(3,4-dihydroxyphenyl)hepta-1,6-diene-3,5-dione.

### Embodiment 22

A process according to any of the previous embodiments for the manufacture of 3,5-bis[(E)-2-(4-hydroxyphenyl)vinyl]phenol (sometimes also referred to as (E,E)-3,5-bis(4′-hydroxystyryl)phenol).

### Embodiment 23

A process according to any of the previous embodiments, which is carried out in at least one solvent, preferably selected from the group, consisting of N-methyl-2-pyrrolidone (NMP), polyethylene glycol (PEG), acetonitrile, dimethylsulfoxide (DMSO), dipropyleneglycol, water and dimethylformamide (DMF), and in the presence of at least one base, preferably selected from amines and basic alkali metal or basic alkaline earth metal compounds, such as acetates, carbonates, hydrogen phosphates, phosphates, in particular sodium acetate, potassium carbonate, potassium phosphate, potassium dihydrogenphosphate.

### Embodiment 24

A process according to any of the previous claims, which is carried out in at least one solvent, preferably selected from the group, consisting of N-methyl-2-pyrrolidone (NMP), polyethylene glycol (PEG), acetonitrile, water and dimethylformamide (DMF), and in the presence of at least one base, preferably sodium acetate.

### Embodiment 25

A process according to any of the previous embodiments, which is carried out in at least one solvent, preferably selected from the group, consisting of N-methyl-2-pyrrolidone (NMP), polyethylene glycol (PEG), acetonitrile, water and dimethylformamide (DMF).

### Embodiment 25

A process according to any of the previous embodiments, which is carried out in at least one solvent, selected from the group consisting of N-methyl-2-pyrrolidone (NMP), acetonitrile and water, or mixtures thereof.

### Embodiment 26

A process according to any of the previous embodiments, which comprises the step of adding water to the process. This embodiment comprises a step of actively adding water to the reaction different from the situation where water is formed during the process.

### Embodiment 27

A process according to any of the previous embodiments, which is carried out in the presence of at least one base, preferably sodium acetate.

### Embodiment 28

A process according to any of the previous embodiments, which is carried out in the presence of at least one phase transfer catalyst compound, preferably quaternary ammonium salts such as tetra-n-butylammonium bromide, methyltrioctylammonium chloride, benzyltrimethylammonium chloride, benzyltriethylammonium chloride, methyltricaprylammonium chloride, methyltributylammonium chloride, and methyltrioctylammonium chloride. Organic phosphonium salts may be also used, e.g., hexadecyltributylphosphonium bromide. Most preferred is tetra-n-butylammonium bromide. The exact mechanism of the phase transfer catalyst compound in the claimed process is not yet known. It was found that its addition can compensate the loss in yield if no phosphane or phosphine is used as a catalyst ligand. So the phase transfer catalyst compound may interact with the transition metal catalyst. Accordingly, in the present invention, the term phase transfer catalyst compound is to be understood that it shall cover known phase transfer catalysts compounds, but it does not necessarily require that the specific phase transfer catalyst compounds actually act as a phase transfer catalyst in process of the invention.

### Embodiment 29

A process according to any of the previous embodiments, preferably according to embodiment 28, which is carried out in the absence of triphenylphosphane, preferably in the absence of phosphanes.

### Embodiment 30

A process according to any of the previous embodiments, which is carried out in the presence of at least one radical scavenger such as 2,6-di-tert-butyl-4-methylphenol (BHT), hydroquinone etc.

### Embodiment 31

A process according to any of the previous embodiments, which is carried out at a temperature of at least 80° C.

### Embodiment 32

A process according to any of the previous embodiments, which is carried out at a temperature in a range between 80° C. to 200° C.

### Embodiment 33

A process according to any of the previous embodiments, which further comprises at least one subsequent derivatization reaction of the compound of formula (I), preferably selected from the group consisting of hydrogenation, esterification, etherification, and salt formation.

### Embodiment 34

A process according to any of the previous embodiments, which further comprises at least one hydrogenation reaction to form hydrogenated derivatives of the formula:

Z—(CH₂—CH₂—Ar)ₐ (I″)

wherein Z, Ar and a are as defined above, preferably a being 2.

### Embodiment 35

A process according to any of the previous embodiments, which further comprises the admixture of a compound as obtained in any of these claims with at least one pharmaceutical or cosmetic excipient.

### EXAMPLES

### Example 1

### Synthesis of (E,E)-3,5-bis(4′-hydroxystyryl)phenol (or 3,5-bis[(E)-2-(4-hydroxyphenyl)vinyl]phenol)

Literature:

A) Green Chem, 2014, 16, 3089: “Preparation of Functional Styrenes from Biosourced Carboxylic acids by Copper Catalyzed decarboxylation in PEG”

B) J. Am. 2002, 124, 11250-51: “Development of a decarboxylative Palladation Reaction and Its Use in a Heck-type olefination of arenes carboxylate”

Reactants:

1) 39.2 g (0.24 mol) of p-coumaric acid

2) 1.04 g Cu(OH)₂

3) 1.2 g of 1,10-phenanthroline

4) 25.7 g (0.1 mol) of 3,5-dibromophenol (Fa. TCl)

5) 0.034 g of Pd(OAc)₂ (Palladium(II)-actetate)

6) 6.0 g Triphenylphosphan

7) 16.4 g of sodium acetate (0.2 mol)

8) 30 g water, demineralized

9) 30 g of acetonitrile

10) 20 g of N-methylpyrrolidone

Procedure

In a 500 ml three-necked flask, the components are successively weighed and the greenish suspension is gassed with nitrogen with stirring for 0.5 hours at room temperature to prevent the oxidation of triphenylphosphine by the dissolved air oxygen. Then the mixture is slowly heated with stirring with a Dean Stark water separator. Initially the acetonitrile and then slowly the water is distilled off. The mixture turns yellow in 2 hours and reaches about 100° C., and then slowly begins to foam (decarboxylation). Up to 120° C., which is reached after a further hour, the reaction mixture becomes red-brown. It is held for another 3 hours at 140° C. until the evolution of gas subsides.

Further processing:

After cooling the reaction mixture is neutralized with 200 ml of 10% hydrochloric acid and with is extracted three times with 100 ml of MTBE (methyl tert-butyl ether). Initially the water phase is bluish later brown. Usually a sugary sticky greenish-yellow precipitate is formed which can be removed with ethyl acetate again. Presumably it is triphenylphosphane oxide (TPPO).

The combined now yellow-brown organic phases are dried over sodium sulfate, filtered and concentrated on a rotary evaporator.

This gives about 55 g of crude product, which contains traces of acetic acid, MTBE and TPPO.

When drying in a drying oven (50 mbar, 50° C.) and then over phosphorus pentoxide in a desiccator under an oil pump vacuum (0.5 mbar) about 49 g of a dark yellow solid foam are obtained that contains the product in about 90% purity (estimated with NMR).

During evaporation of a solution with ethyl acetate yellow crystals are sometimes formed. Or a crystallization can be induced by using such crystals in a highly viscous crude product.

In the purification by flash chromatography with a cyclohexane / ethyl acetate gradient further purification to purities of above 95% can be achieved.

Further examples 2 to 8 are carried out as in example 1 with the amounts of reactants shown in the following table.

Therein the mol-% values for Cu(OH)₂ and 1,10-phenanthroline are based on the molar amounts of p-coumaric acid.

The mol % values for palladium(II)acetate, triphenylphosphine (triphenylphosphane), tetra-n-butylammonium bromide, sodium acetate, potassium carbonate and 2,6-di-tert-butyl-4-methylphenol BHT are based on the molar amount of the 3,5-dibromophenol used.

| | Example | | | | | | | | | | | | | |
|---|---|---|---|---|---|---|---|---|---|---|---|---|---|---|
| | 2 | | 3 | | 4 | | 5 | | 6 | | 7 | | 8 | |
| Reactant | | | | | | | | | | | | | | |
| p-coumaric acid | 28 | mmol | 28 | mmol | 28 | mmol | 28 | mmol | 28 | mmol | 28 | mmol | 28 | mmol |
| copper(II) hydroxide | 1.78 | mol % | 1.78 | mol % | 1.78 | mol % | 1.78 | mol % | 1.78 | mol % | 1.78 | | 1.78 | |
| 1,10-phenanthroline | 1.78 | mol % | 1.78 | mol % | 1.78 | mol % | 1.78 | mol % | 1.78 | mol % | 1.78 | | 1.78 | |
| 3,5-dibromophenol | 10 | mmol | 10 | mmol | 10 | mmol | 10 | mmol | 10 | mmol | 10 | mmol | 10 | mmol |
| palladium(II)acetate | 0.6 | mol % | 0.6 | mol % | 0.6 | mol % | 0.6 | mol % | 0.6 | mol % | 0.6 | mol % | 0.6 | mol % |
| triphenylphosphine | | | | | | | 38 | mol % | | | | | | |
| (triphenylphosphane) | | | | | | | | | | | | | | |
| tetra-n-butylammonium bromide | 30 | mol % | 30 | mol % | 30 | mol % | | | | | 24 | mol % | 24 | mol % |
| sodium acetate | | | | | | | 240 | mol % | — | | | | | |
| potassium carbonate | 200 | mol % | 200 | mol % | 200 | mol % | 200 | mol % | 200 | mol % | | | 200 | mol % |
| water | 3 | g | — | | — | | 3 | g | 3 | g | 3 | g | 3 | g |
| 2,6-di-tert-butyl-4-methylphenol | 5 | mol % | 5 | mol % | 0.5 | mol % | | | 5 | mol % | 5 | mol % | 5 | mol % |
| BHT | | | | | | | | | | | | | | |
| acetonitrile | | | | | | | 9 | g | | | | | | |
| N,N-dimethylformamide | 9 | g | 9 | g | 9 | g | | | 9 | g | 9 | g | 9 | g |

| | | | | | | | |
|---|---|---|---|---|---|---|---|
| Yield | 94.84% | 59.69% | 44.84% | 97.57% | 41.21% | 73% | 67.27% |

The examples 2 and 5-8 show that the process according to the invention leads to higher yields if water is added to the process.

While with triphenylphosphine high yields were obtained, separating the resulting triphenyl phosphine oxide from the product can be difficult. However, working in the presence of a phase transfer catalyst compound such as tetra-n-butylammonium bromide can almost compensate the absence of the triphenyl phosphine and avoids the formation of triphenyl phosphine oxide and its undesirable separation from the product.

1. A process for the manufacture of a hydroxy-substituted aromatic compound of the formula (I):
Z—(CH═CH—Ar)ₐ (I)
wherein
Z is a divalent substituted aromatic group or a divalent group of the formula: 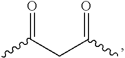
wherein [symbol] denotes a single bond,
Ar independently is selected from a substituted aromatic group, and a is 2,
or a salt thereof,
which comprises reacting a compound of the formula (II):
Z—(X)ₐ (II)
wherein
X is a leaving group and
Z and a are as defined above,
with a compound of formula (III):
CH₂═CH—Ar (III)
wherein Ar is as defined above,
in the presence of a transition metal catalyst,
with the proviso that the group Z and the group Ar each are substituted by at least one hydroxy group.

2. The process according to claim 1, wherein Z is a divalent substituted aromatic group.

3. The process according to claim 1, wherein the compound of formula (III) is formed in situ from a compound of formula (IV)
HOOC—CH═CH—Ar (IV),
wherein Ar is as defined above.

4. The process according to claim 1, wherein the transition metal catalyst is a bimetallic catalyst comprising palladium and at least one further transition metal.

5. The process according to claim 1, wherein the leaving group X is a halogenide.

6. The process according to claim 1, wherein Z is derived from a substituted six-membered aromatic group.

7. The process according to claim 1, wherein Z is derived from a hydroxy-substituted benzene group and/or Ar is derived from a hydroxyl-substituted benzene group.

8. The process according to claim 1, wherein each group Ar is derived from a hydroxy-substituted benzene group.

9. The process according to claim 1, wherein the compound of formula (I) is selected from the group consisting of:

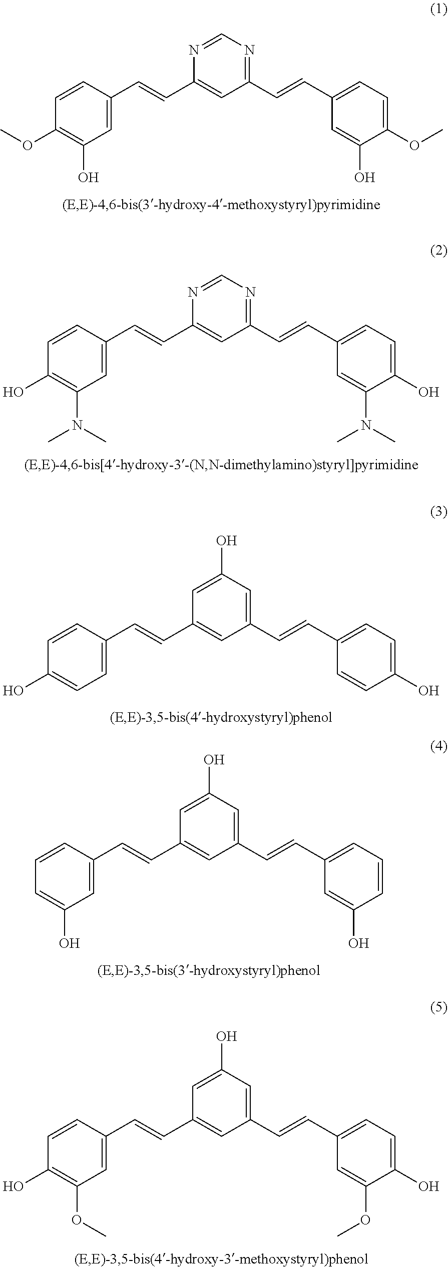

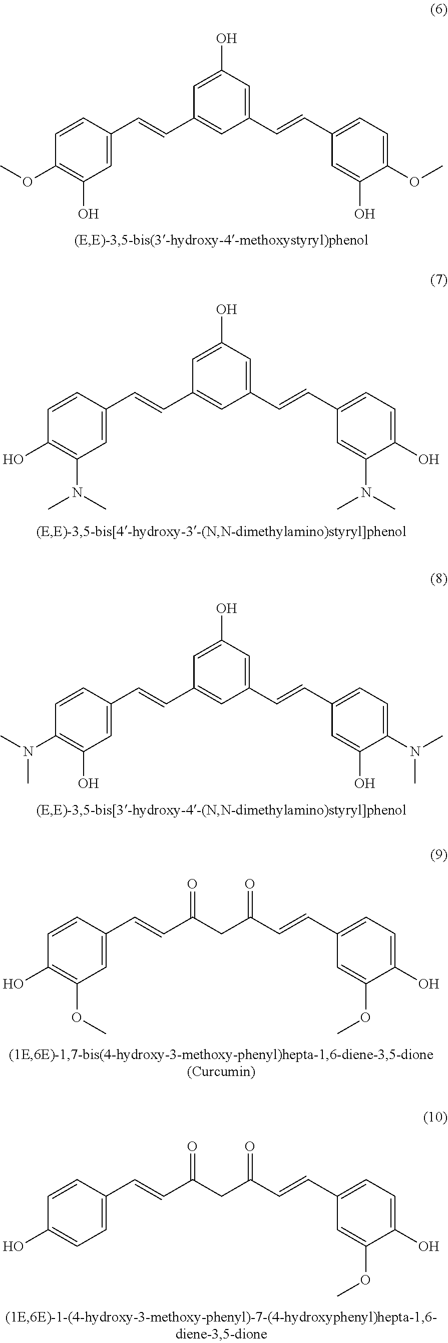

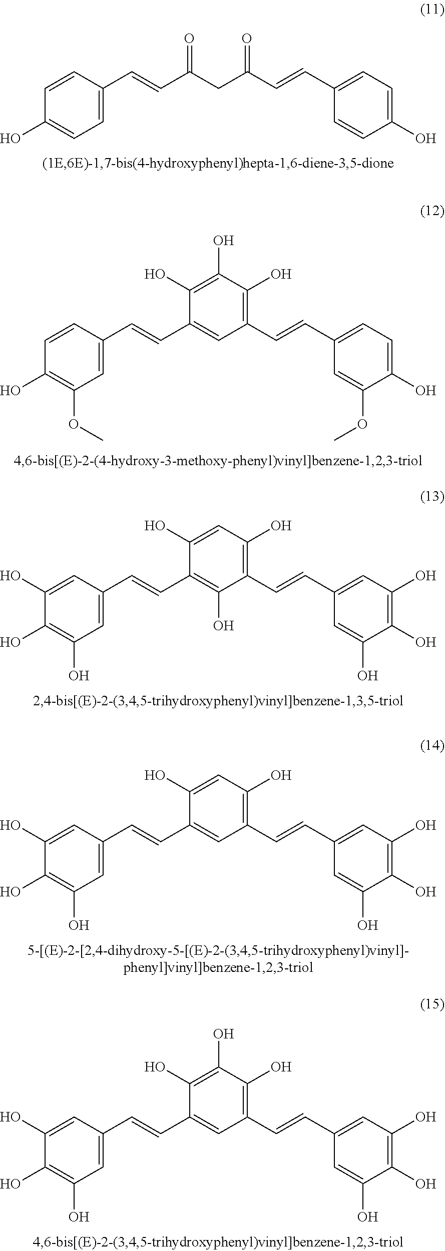 [SMILES: O=C(/C=C/c1ccc(O)cc1)CC(=O)/C=C/c1ccc(O)cc1 ; COc1cc(/C=C/c2cc(/C=C/c3ccc(O)c(OC)c3)c(O)c(O)c2O)ccc1O ; Oc1cc(/C=C/c2c(O)cc(O)c(/C=C/c3cc(O)c(O)c(O)c3)c2O)cc(O)c1O ; Oc1cc(O)c(/C=C/c2cc(O)c(O)c(O)c2)cc1/C=C/c1cc(O)c(O)c(O)c1 ; Oc1cc(/C=C/c2cc(/C=C/c3cc(O)c(O)c(O)c3)c(O)c(O)c2O)cc(O)c1O]

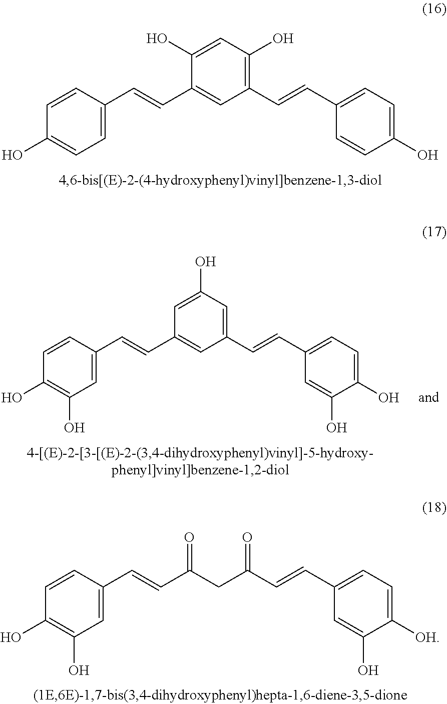 [SMILES: Oc1ccc(/C=C/c2cc(/C=C/c3ccc(O)cc3)c(O)cc2O)cc1 ; Oc1cc(/C=C/c2ccc(O)c(O)c2)cc(/C=C/c2ccc(O)c(O)c2)c1 ; O=C(/C=C/c1ccc(O)c(O)c1)CC(=O)/C=C/c1ccc(O)c(O)c1]

10. The process according to claim 1 for the manufacture of a compound of the formula:

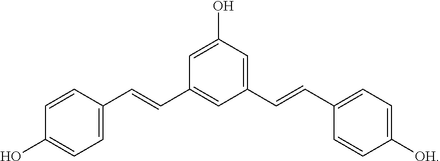 [SMILES: Oc1ccc(/C=C/c2cc(O)cc(/C=C/c3ccc(O)cc3)c2)cc1]

11. The process according to claim 1, which is carried out in at least one solvent, and in the presence of at least one base.

12. The process according to claim 1 comprising adding water to the process.

13. The process according to claim 1, which is carried out in the presence of at least one phase transfer catalyst compound.

14. The process according to claim 13, which is carried out in the absence of triphenylphosphane.

15. The process according to claim 1, which further comprises at least one subsequent derivatization reaction of the compound of formula (I).

16. The process according to claim 1, which further comprises the admixture of a compound of formula (1) with at least one pharmaceutical or cosmetic excipient.

17. The process according to claim 4, wherein the at least one further transition metal comprises copper or silver.

18. The process according to claim 5, wherein the halogenide is chlorine or bromine.

19. The process according to claim 6, wherein the substituted six-membered aromatic group is benzene, pyridine, or pyrimidine.
